(19) United States
(12) Patent Application Publication
(10) Pub. No.: US 2023/0001075 A1
(43) Pub. Date: Jan. 5, 2023

(54) ANTI-TWIST BEARING OF A RETENTION ARM OF A MEDICAL SYRINGE PUMP

(71) Applicant: B. Braun Melsungen AG, Melsungen (DE)

(72) Inventors: Jan Schwarz, Melsungen (DE); Hans-Josef Gerlach, Marsberg (DE); Stefan Batzdorf, Fulda (DE); Marcel Stich, Nieste (DE); Matthias Schwalm, Schwalmstadt (DE); Joachim Schuetz, Fulda (DE)

(21) Appl. No.: 17/851,112

(22) Filed: Jun. 28, 2022

(30) Foreign Application Priority Data
Jul. 1, 2021 (DE) 20 2021 103 539.0

(51) Int. Cl. A61M 5/145

(52) U.S. Cl. CPC A61M 5/1452; A61M 2005/1401

(57) ABSTRACT

A medical syringe pump includes a housing and a drive head movable linearly to the housing by a tube-shaped or rod-shaped retention arm for actuating a syringe inserted in the housing, for which purpose the retention arm is connected in an axially fixed manner with an advance carriage and is in engagement with an advance spindle via the advance carriage. The medical syringe pump includes a twist protection which protects the advance carriage from twisting about the axis of the retention arm.

### CROSS-REFERENCE TO RELATED APPLICATION

This application claims priority under 35 U.S.C. § 119 to German Application No. 20 2021 103 539.0, filed Jul. 1, 2021, the content of which is incorporated by reference herein in its entirety.

### FIELD

The disclosure relates to a medical syringe pump with a linearly movable drive head for actuating a syringe inserted in the housing with an anti-twist bearing of a retention arm of said medical syringe pump.

### BACKGROUND

The development in modern medicine, especially in intensive-care medicine, has led to infusion therapies requiring the specific use and the exact dosing of highly efficient drugs. One possibility of this exact dosing are medical syringe pumps into which a syringe can be inserted in a defined manner. The syringe pump ensures that the syringe is actuated automatically and precisely to effect a defined temporal dosing of a medical substance. A prerequisite of an orderly actuation of the syringe is that both the syringe cylinder and the syringe piston are fixed and guided properly within the syringe pump.

A conventional medical syringe pump for dosing a liquid contained in a syringe at a defined rate is, for instance, known from DE 298 12 065 U1. Here, the syringe is pressed over a piston rod by means of a piston, and the syringe pump is designed such that it detects the size of the syringe inserted by means of a force sensor.

Furthermore, EP 1 374 932 A2 discloses a medical syringe pump with a housing comprising a syringe depression for a syringe to be inserted. In addition to the housing the syringe pump comprises a linearly movable drive head for actuating the syringe piston of the syringe.

Conventionally, medical syringe pumps comprise a drive rod/retention arm which projects from the housing of the syringe pump and by which a linear movement of the drive head and of the clamped syringe piston is enabled. The drive rod is mounted with an axially fixed portion of the syringe pump, especially a rod-shaped spindle. Twisting of this drive rod and hence of the drive head connected therewith can, however, not be prevented completely by such an axial bearing, so that a proper fixing of the syringe is not fully guaranteed.

### SUMMARY

It is therefore an object of the present disclosure to avoid or at least mitigate the afore-described disadvantages. For this purpose, a syringe pump with a linearly movable retention arm which is protected from twisting about its own axis is to be provided.

The disclosure accordingly relates to a medical syringe pump with a housing and a drive head (corresponds to the part getting into contact with a syringe piston rod) movable linearly to the housing via a tube-shaped or rod-shaped retention arm for actuating a syringe inserted in the housing, for which purpose the retention arm is connected in an axially fixed manner with an advance carriage which is in turn in meshing engagement with an advance spindle. The medical syringe pump comprises a twist protection and/or twist protection component formed separately from the advance carriage and protecting the advance carriage from twisting about the axis of the retention arm.

In other words, a separately formed twist protection is connected with the advance carriage in a medical syringe pump such that it is mounted/retained in an anti-twist manner in the pump housing. The advance carriage is connected with the retention arm in an axially fixed manner, i.e. in a shift-proof manner along the retention arm axis, so that the retention arm is also protected from twisting by means of the twist protection. This retention arm is driven by means of the advance spindle (extending in parallel to the retention arm) with which the retention arm is in engagement via the advance carriage. Engagement takes place substantially via a threaded bearing (internal thread) integrated in the advance carriage, so that the retention arm is movable along the axis of the advance spindle in the case of a rotary motion of the advance spindle. The retention arm comprises, at an end portion thereof, a drive head contactable with a syringe piston rod, which actuates, by the linear movement of the retention arm, a syringe inserted in the syringe pump by means of pushing in and/or pulling out of the syringe piston.

Due to such a twist protection the linearly movable retention arm is protected from twisting relative to the pump housing, so that an additional (guiding) stability of the retention arm and the drive head connected therewith is ensured. Thus, a proper fixing and operation of a syringe is fully guaranteed. It is, for instance, possible to efficiently avoid canting of the syringe piston, for example, due to twisting of the retention arm and a resulting pivoting of the drive head.

In accordance with a further preferred aspect the twist protection of the medical syringe pump in accordance with the disclosure is of clamp-shaped design and comprises a gate-shaped or U-shaped lateral profile as well as a plurality of feet engaging the advance carriage in a form-fit manner.

In other words, a plurality of elongate, especially parallel, feet/legs/pins is formed as a part of the clamp-shaped twist protection, which is why the twist protection comprises a gate-shaped and/or U-shaped lateral profile. In the mounted state of the, preferably single-part, twist protection these feet engage/stick in a form-fitting manner in the advance carriage fixed to the retention arm.

In accordance with a further preferred aspect the twist protection comprises four feet which are, in the mounted state of the twist protection, oriented substantially perpendicular to the axis of the retention arm and encompass same.

In other words, the twist protection comprises four feet arranged in a rectangular shape and formed in parallel to each other and perpendicular with respect to the axis of the retention arm. Furthermore, the four feet are formed such that, in the mounted state of the twist protection, every two of the feet are arranged at one side of the retention arm axis and encompass the retention arm substantially transversely to the axis and/or circumferentially.

This kind of design of the twist protection can be produced by a simple and cost-efficient manufacturing method and can be mounted easily and safely to the advance carriage by the form-fitting engagement of the feet.

In accordance with a further preferred aspect the retention arm of the medical syringe pump comprises circumferential grooves which are designed and arranged such that at least two feet of the twist protection engage therewith in the mounted state.

In other words, grooves extending circumferentially in a respective plane of the retention arm are arranged at the retention arm of the medical syringe pump, and receive at least two, especially parallel, feet of the mounted twist protection.

These grooves at the retention arm serve for an additional stabilizing and/or guiding function of the feet of the twist protection in the mounting process and/or in the mounted state.

In accordance with a further preferred embodiment the advance carriage of the medical syringe pump comprises recesses which are formed and arranged to receive the feet of the twist protection.

In other words, recesses/slits/openings are provided at the advance carriage in which the feet of the twist protection engage and/or can be received. Preferably, the feet of the twist protection engage the recesses of the advance carriage in an undercutting manner.

In accordance with a further preferred aspect the feet of the twist protection are designed such that, in the mounted state of the twist protection, a clearance or a gap exists between the feet of the twist protection and the lower side of the housing of the syringe pump, so that an axial shifting of the retention arm, but no twisting of the retention arm about its axis is possible.

In other words, the feet of the twist protection have such a length and basis that they are, in the mounted state of the twist protection, movable axially, i.e. in the direction of the retention arm axis, along the inner lower side of the housing or possibly along the inner upper side of the housing of the syringe pump in a sliding/slipping manner without the advance carriage connected with the twist protection being able to be twisted.

This precisely adapted clearance between the feet of the twist protection and the lower side of the housing of the syringe pump ensures a smooth axial shifting of the retention arm as well as a safe protection from twisting, so that a precise and stable as well as proper actuating of the syringe within the syringe pump is guaranteed.

In accordance with a further preferred aspect the twist protection comprises a through-hole oriented in parallel to the feet of the twist protection, which is formed at an arc portion of the clamp-shaped twist protection that connects the feet, and in which a locking screw is screwed which retains the twist protection at the advance carriage from unintended disengaging.

In other words, the twist protection comprises an arc portion which is substantially of cuboid design and from which the feet protrude/project in parallel, preferably at the corner points of the arc portion. At this arc portion, in parallel to the feet, preferably centrally, a through-hole is provided in which a locking screw can be received, and by which the twist protection is held together with the advance carriage which preferably also comprises a through-hole.

### BRIEF DESCRIPTION OF THE DRAWING FIGURES

The subject matter disclosed will now be explained in detail by means of advantageous embodiments and with reference to the associated Figures. The Figures are of schematic nature only and serve only for the understanding of the disclosure. It is pointed out that the features of different embodiments are interchangeable and may occur in any combination.

FIG. 1 is an isometric view of an operating position of the retention arm with the advance carriage and the advance spindle of the medical syringe pump, wherein the twist protection is illustrated in an exploded view.

FIG. 2 is a sectional view through the advance carriage and the twist protection in a mounted state.

FIG. 3 is a sectional view through the axis of the advance spindle and shows the advance carriage and the twist protection in a mounted state.

### DETAILED DESCRIPTION

In the following, the present disclosure and the advantageous embodiments will be described by means of the Figures.

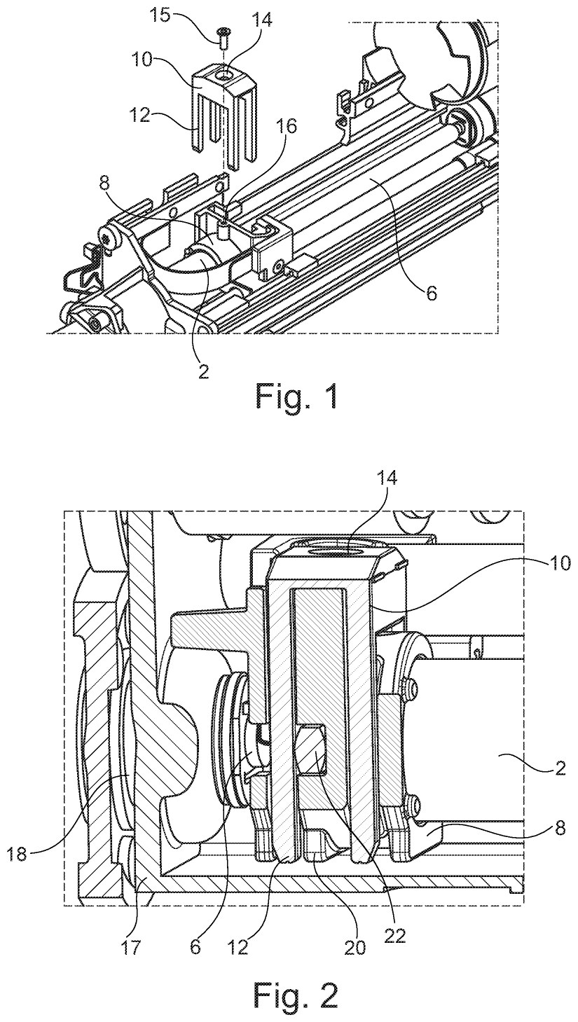

FIG. 1 is an isometric view of an operating position of the retention arm 2 in the housing of the medical syringe pump in accordance with the disclosure. The retention arm 2 is guided via a rod-shaped advance spindle 6 whose longitudinal axis concurs with the longitudinal axis of the tube-shaped retention arm 2. The retention arm 2 is, substantially via an advance carriage 8, mounted axially with the advance spindle 2 and/or guided by same. The advance carriage 8 is arranged at an end of the retention arm 2 which faces away from the drive head and encompasses same. The advance carriage 8 is connected in an axially fixed manner with the retention arm 2 and the retention arm 2 is, in this view, in a substantially extended state, i.e. in a position in which the advance spindle 6 projects from the retention arm 2. Furthermore, FIG. 1 shows the clamp-shaped twist protection 10 which comprises a substantially cuboid basis and four feet 12. The four feet 12 of the twist protection 10 are each formed at the corners of the basis of the basic body and project perpendicularly to this basis. The feet 12 have a constant cross-section and a rectangular layout. Furthermore, the advance carriage 8 and the clamp-shaped twist protection 10 both comprise a vertical through-hole 14, 16 which are arranged such that the advance carriage 8 and the twist protection 10 are connectable by a locking screw 15.

FIG. 2 is a sectional view through the advance carriage 8 and the twist protection 10 in a mounted state. The sectional plane extends in parallel along the longitudinal axis of the retention arm 2 of the syringe pump and intersects the advance carriage 8 and two feet 12 of the clamp-shaped twist protection 10. In this view the retention arm 2 of the syringe pump is slid substantially completely over the advance spindle 6, i.e. the retention arm 2 is in a retracted position. In this position of the retention arm 2 the advance carriage 8 and the twist protection 10 fastened thereto are arranged next to a perpendicular housing wall 17 of the syringe pump which comprises a gearing 18 for the advancing of the retention arm 2. The basic body of the advance carriage 8 is of hollow cylinder-shaped design and encloses the retention arm 2 of the syringe pump. At the side facing the basis of the syringe pump, the advance carriage 8 comprises three parallel, substantially skid-shaped projections 20. These projections 20 are of flat design at their underside, so that the advance carriage 8 slides at the inner side of the basis of the syringe pump when the retention arm 2 of the syringe pump performs a linear movement. The four feet 12 of the twist protection 10 extend, in the mounted state, in parallel along the perpendicular housing wall 17 of the syringe pump and engage in vertical recesses of the advance carriage 8 which are adapted to the dimensions of the feet 12 of the twist protection 10. Every two of the feet 12 are arranged at one side of the longitudinal axis of the retention arm 2, i.e. the feet 12 of the twist protection 10 encompass substantially the retention arm 2. Moreover, the twist protection 10 comprises, in this view at the topmost end, a through-hole 14.

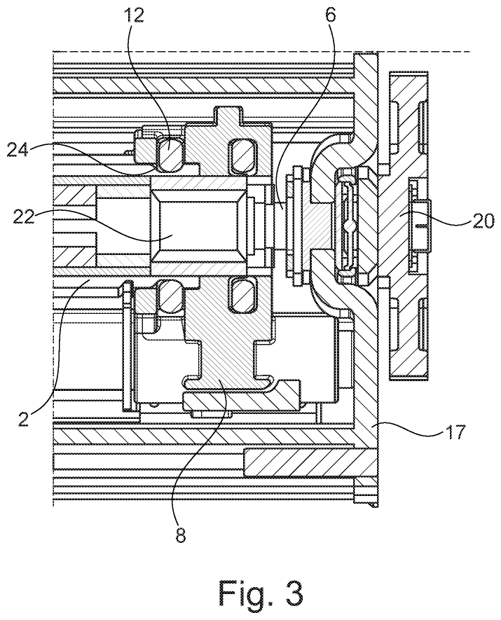

FIG. 3 is a sectional view through the axis of the advance spindle 6 and shows the advance carriage 8 and the twist protection 10 in a mounted state, wherein the sectional view has been taken from the underside of the syringe pump. In this view the advance spindle 6 and the retention arm 2, which has a larger inner diameter than the outer diameter of the advance spindle 6, extend horizontally. The retention arm 2 is connected with the advance spindle 6 via a threaded bearing 22 of the advance carriage 8. A gearing 18 which is partially arranged externally of the syringe pump housing drives the advance spindle 6, so that a linear movement of the retention arm 2 is performed via the threaded bearing 22. In the sectional plane illustrated the retention arm 2 encloses a portion of the threaded bearing 22 of the advance carriage 8, which consequently projects partially from an end of the retention arm 2. The layouts of the four feet 12 of the twist protection 10 are illustrated in this sectional view and engage in recesses of the advance carriage 8 which are provided for the feet 12. In this view the feet 12 of the twist protection 10 are arranged such that every two of the feet 12 are arranged at one side of the axis of the retention arm 2, wherein the axis of the retention arm 2 forms an imaginary mirror axis. The two feet 12 closer to the gearing 18 are, in this view, completely enclosed by the advance carriage 8. The two remaining feet 12 of the twist protection 10 rest, in this view, against the advance carriage 8 with their long layout sides, and the short layout side which is closer to the axis of the retention arm 2 rests against the retention arm 2. For this purpose, the retention arm 2 comprises two lateral grooves 24 in which these two feet 12 engage and abut.

1. A medical syringe pump comprising:
a housing;
a drive head;
a retention arm that is tube-shaped or rod-shaped and defining an axis;
and
a twist protection,
the drive head being movable linearly relative to the housing by the retention arm for actuating a syringe inserted in the housing,
the retention arm being connected in an axially fixed manner with an advance carriage,
the retention arm being in engagement with an advance spindle, and
the twist protection preventing the advance carriage from twisting about the axis of the retention arm.

2. The medical syringe pump according to claim 1, wherein the twist protection is of clamp-shaped design and comprises a gate-shaped or U-shaped lateral profile as well as a plurality of feet engaging the advance carriage in a form-fitting manner.

3. The medical syringe pump according to claim 2, wherein the twist protection comprises four feet which are, in a mounted state of the twist protection, oriented substantially perpendicular to the axis of the retention arm and encompass the retention arm.

4. The medical syringe pump according to claim 2, wherein the retention arm comprises circumferential grooves which are formed and arranged such that at least two of the plurality of feet of the twist protection engage in the grooves in a mounted state of the twist protection.

5. The medical syringe pump according to claim 2, wherein the advance carriage comprises recesses which are formed and arranged such that they receive the plurality of feet.

6. The medical syringe pump according to claim 2, wherein the plurality of feet are formed such that, in a mounted state of the twist protection, a clearance or a gap exists between the plurality of feet and a lower side of the housing to permit an axial shifting of the retention arm but prevent twisting of the retention arm about the axis of the retention arm.

7. The medical syringe pump according to claim 2, wherein the twist protection comprises a through-hole oriented in parallel to the plurality of feet of the twist protection, wherein the through-hole is formed at an arc portion of the twist protection that connects the plurality of feet, wherein a locking screw is screwed into the through-hole to retain the twist protection against unintended disengaging at the advance carriage.
